(19) United States
(12) Patent Application Publication
(10) Pub. No.: US 2022/0218501 A1
(43) Pub. Date: Jul. 14, 2022

(54) SENSOR SUPPORT FOR ARRANGING ON A PROSTHESIS

(71) Applicant: Saphenus Medical Technology GmbH, Krems an der Donau (AT)

(72) Inventors: Rainer Schultheis, Wien (AT); Bernhard Zach, Wien (AT); Martin Brandstätter, Wien (AT); Aaron Pitschl, Wien (AT); Andreas Mühlenberend, Weimar (DE)

(73) Assignee: Saphenus Medical Technology GmbH, Krems an der Donau (AT)

(21) Appl. No.: 17/296,337

(22) PCT Filed: Nov. 28, 2019
(86) PCT No.: PCT/IB2019/060297

§ 371 Date: Jan. 17, 2022

(30) Foreign Application Priority Data
Nov. 30, 2018 (EP) 18209621.4

(51) Int. Cl. A61F 2/76; A61F 2/70

(52) U.S. Cl. CPC A61F 2/76; A61F 2/66; A61F 2/70

(57) ABSTRACT

The invention relates to a sensor support (10) for arranging on a prosthesis, comprising a main part (20) for receiving sensors, said main part (20) having a sensor section (25) comprising sensors. The main part (20) has a holding section (30) for holding the main part (20) on a prosthesis part and a knitted fabric tube (21). The holding section (30) has a first knitted structure (32) which allows a secure hold of the main part (20) on the prosthesis part, and the sensor section (25) has a different knitted structure (27) which allows a secure positioning of the sensors relative to the prosthesis part.

The invention relates to a sensor support for arranging on a prosthesis according to the preamble of claim 1.

Modern prostheses are often equipped with sensors. Sensors are typically arranged directly inside a prosthesis and are permanently fitted therein. For example, multiple pressure sensors are fitted inside the prosthetic sole of a prosthetic foot. The prosthesis often includes several stimulators as well. These stimulators are electrically connected to multiple pressure sensors. The stimulators are thus controlled and excited by the pressure sensors, and act on the patient's remaining extremity through the skin on. In this way, the stimulators enable the prosthesis user to perceive a pressure on the pressure sensors and thus “feel” the pressure on the prosthetic sole. Apparatuses of such kind are disclosed in WO 98/25552 A1 for example.

The disadvantage of this known solution is that not only are prosthesis systems of such kind very expensive, the adaptation phase and familiarisation phase in which the prosthesis user gets used to the prosthesis take a great deal of time. Furthermore, they also entail multiple appointments with a specialist doctor and/or orthopaedist. There, the positions of the stimulators and pressure sensors must be adjusted several times, which can be difficult if they are already fitted permanently.

It is the object of the present invention, to remedy one or more drawbacks of the related art. In particular, a sensor support is to be produced which can be attached to existing prostheses easily without changing them structurally. In the fitted state on prosthesis, the sensor support should function reliably, but on the other hand it should also be easy to remove and fitted again in another prosthesis, for example.

This object is solved by the features of the independent claim. Advantageous further developments are presented in the description, the figures, the description of the drawing, and in the dependent claims.

A sensor support according to the invention for arranging on a prosthesis comprises a main part for receiving sensors, wherein said main part has a sensor section with sensors, and the main part has a holding section for holding the main part on a prosthesis part. The main part includes a knitted fabric tube. The holding section has a first knitted structure, which allows a secure hold of the main part on the prosthesis part. The sensor section has a different knitted structure which allows a secure positioning of the sensors relative to the prosthesis part.

The present sensor support can be arranged on any prosthesis, which accordingly is not required to have any sensors itself and may therefore be of simpler construction. In this arrangement, not only are the sensors positioned fixedly in the main part, but the sensors are also fixed in a previously defined position on the prosthesis part with the aid of the different knitted structures on the sensor section and the holding section of the main part. The knitted structure on the adhesive section prevents creases from forming in the fabric tube. Creasing might result in incorrect positioning of the sensors on the prosthesis. The knitted structure on the sensor section knitted relatively more sturdily and thus minimises the mechanical load on the sensor when the prosthesis is in use, thereby prolonging its service life, and the prosthesis itself is exposed to less impact loading (damping).

The novel sensor support enables the prosthesis user to “feel” the prosthetic motion/rolling motion of the prosthetic foot, as with conventional prostheses with built-in sensors, but it also allows easy removability of the sensors together with the sensor support, simple cleaning of the prosthesis, thus improving daily interaction with the prosthesis.

Even difficult movements, such as moving/walking on steep terrain with an artificial foot, can be learned using the novel sensor support. Since this sensor support can be arranged on any prosthesis of any kind, the prosthesis user does not have to change the prosthesis if he/she wishes to use a “feeling” prosthesis. Since replacing a prosthesis is highly uncomfortable for prosthesis user, because the prosthesis shaft must be adapted to prosthesis user's remaining anatomy, the invention is extremely advantageous.

In particular, the sensor support is suitable for arrangement on an artificial hand or an artificial foot. These are widely used prosthesis types, which are thus able to be improved with the sensor support as described herein.

The holding section preferably includes a second knitted structure in addition to its first knitted structure. The knitted structures on the holding section enable a secure hold on the prosthesis part. They are arranged in various positions on the main part, so the main part cooperates with the prosthesis part with stable holding effect in more than one position.

The second knitted structure is preferably designed differently from the first knitted structure. Different knitted structures have different stretch characteristics, enabling the sensor support to be arranged reliably on the prosthesis part.

In particular, the second knitted structure is of dimensionally stable design in order to enclose a prosthesis part. A dimensionally stable knitted structure creates an improved hold on the prosthesis part, as the knitted structure is connected to the prosthesis part in force-fitting manner. For example, the dimensionally stable knitted structure is arranged on the area of the sensor support which cooperates with the heel of an artificial foot. The affords the prosthesis user the improved “feel” of a sequence of movements, when walking or running, for example, thereby increasing the prosthesis user's confidence in the prosthesis and consequently their stability when walking.

More preferably, besides its first knitted structure the holding section also includes a third knitted structure, which is designed differently from both the first knitted structure and the second knitted structure. This serves to further improve the holding properties of the sensor support on the prosthesis by preventing the formation of creases in the knitted fabric tube.

In particular, the third knitted structure is of elastic construction. The knitted fabric tube may be pre-tensioned and may be stretched over the prosthesis part easily with the aid of the elastic third knitted structure.

Preferably, the first knitted structure of the holding section has a first denier, and the second knitted structure of the holding section has a second denier auf. The different knitted structures described here have different functionalities, which can be adjusted on the one hand through the knitted structure itself, but also using the different deniers. The different functionalities of the knitted structure are advantageous in various sections of the prosthesis. The holding properties of identical knitted structures may be improved with the different deniers. For example, a diamond-shaped knitted structure is advantageous, because such structures have favourable stretch characteristics. In particular, the denier of the first knitted structure is in a range between 0.1 mm and 0.6 mm, and is advantageously equal to 0.3 mm. In particular, the denier of the second knitted structure is in a range between 0.5 mm and 1.5 mm, and is advantageously equal to 1 mm.

The third knitted structure of the holding section preferably has a third denier. A third knitted structure with a third denier increases the flexibility of the range of applications for the sensor support.

In particular, the denier of the third knitted structure is in a range between 0.3 mm and 0.9 mm, and is advantageously equal to 0.6 mm.

More preferably, the knitted structure of the sensor section has denier which is different from the first denier of the holding section. In this way, the properties of the knitted structure that are specific to the sensor section are improved. For example, the denier of the knitted structure of the sensor section is thicker than the denier on the holding section, and so the sensors in the sensor section are protected from undesirable mechanical loads. In particular, the denier of the knitted structure of the sensor section is in a range between 0.6 mm and 1.5 mm, and is advantageously equal to 0.8 mm.

Preferably, at least one of the knitted structures is made from a resistant synthetic fibre. Synthetic fibres are durable and robust materials which can be worked in a knitting procedure. For example, a synthetic fibre made from nylon or polyester or materials with comparable properties is used. These serve to prevent the knitted fabric tube from becoming stretched or worn in selected areas. Particularly in the area of a prosthetic sole of an artificial foot, it is particularly important to prevent stretching and compression, in order to protect the sensors from damage by tensile forces.

More preferably, the sensor section has a multilayer construction and has one sensor layer in which the sensors are embedded. The sensor layer enables the sensors to be arranged in the sensor support in structured manner, which also allows provision to be made for the prosthesis user's individual needs. The multilayer structure of the sensor section results in improved protection of the sensor and at the same time contributes to the damping effect between the prosthesis and its surroundings (e.g., the ground or an inner shoe).

As is generally known, the sensors preferably comprise at least one pressure sensor. The at least one pressure sensor is designed for the purpose of recording pressures that normally act directly on the prosthesis and converting them into an electrical signal.

The electrical signal is then forwarded. The at least one pressure sensor functions piezoelectrically, for example.

Alternatively or additionally, the sensors comprise at least one force sensor. The force sensor is designed for the purpose of recording forces that normally act directly on the prosthesis and converting them into an electrical signal, which is then forwarded. A strain gauge is used as a force sensor, for example, because it is lightweight and can be arranged in the sensor layer without taking up much space. Besides compressive forces, force sensors are also designed to measure shearing forces.

An outer protective layer which is farthest from a prosthesis part when in use is preferably provided on the sensor section in addition to the knitted structure. The outer protective layer protects the sensor layer from unwanted mechanical loads, which can lead to the formation of creases in the sensor layer, for example. Consequently, not only can the sensor support be arranged directly on the prosthesis, it can also be used with the prosthesis inside an item of clothing, such as a shoe or glove, which might lead to the formation of creases on the sensor layer without this improved arrangement. In addition, this outer protective layer prolongs the service life of the sensor layer if the user walks on the ground or a floor without additional protective arrangements, for example.

Preferably, an inner protective layer is provided which is closest to a prosthesis part. The inner protective layer is designed for purpose of protecting the sensor layer mechanically from the inside, for example when the sensor support is stretched over the prosthesis. The stretching of the sensor support over the prosthesis may engender stretching forces which are absorbed by the inner protective layer and thus prevented from acting on the sensor layer.

More preferably, the outer protective layer is designed to suppress slipping at least in one direction. This prevents the outer protective layer from slipping on the ground or against an inner shoe, which in turn also prevents the sensor layer from slipping out of position in the sensor support. Synthetic leathers such as Alcantara may be used as the outer protective layer. The surface of the outer protective layer is designed to suppress slipping, for example.

In particular, at least a section of the outer protective layer, or the entire layer, is manufactured from an anti-slip material. An anti-slip material prevents the sensor support from slipping when the prosthesis is moved while it is located together with the sensor support in an item of clothing such as a shoe. In this situation, the anti-slip material interacts frictionally with the material of the item of clothing.

However, the anti-slip material may also be applied as a coating on a support material. In this case, the support material may be dimensionally stable and only the coating may be designed to suppress slipping. For example, a synthetic leather may be used as support material, covered with a coating of silicone or similar materials. Silicone can be applied easily and over large expanses of a support material and has strong adhesive stability, thereby preventing the coating from being worn off too quickly.

Alternatively or additionally, at least a section of the outer protective layer is made from a slip-favouring material. A slip-favouring material enables the outer protective layer to slip readily at least in one direction, thereby making it easier to arranged the sensor support inside an item of clothing, for example. In this arrangement, the slip-favouring material interacts with the item of clothing in a way that favours slipping (at least in one direction).

In particular, the outer protective layer is designed to favour slipping at least in one direction. This simplifies the task of arranging the sensor support inside an item of clothing and prevents the formation of creases on the sensor support. The slip-favouring material may also be applied to support material as a coating. In this case, the support material may be dimensionally stable and only the coating may be designed to favour slipping. For example, a synthetic leather may be used as support material, covered with a coating of vinyl, such as a vinyl film, or similar materials. A vinyl film can be applied easily and over large expanses of a support material and has strong adhesive stability, thereby preventing the coating from being worn off too quickly.

The outer protective layer preferably has a profile structure in order to enhance its anti-slip or slip-favouring property. A profile structure such as is described herein is understood to be a structured arrangement of materials having different properties which are arranged at a structured distance from each other and interact with each other to improve the anti-slip properties in one direction or the slip-favouring property in another direction of the sensor support. This may be a smooth, fur-like structure, for example.

Alternatively or additionally, the inner protective layer is designed to inhibit slipping at least in one direction. This prevents the inner protective layer from slipping out of position and therewith also prevents the sensor layer from slipping in the sensor support. The anti-slip material may be applied to a support material as a coating. In this arrangement, the support material may be dimensionally stable and the coating may be anti-slip. As described previously, for example, a support material made of synthetic leather coated with silicone may be used.

The inner protective layer is preferably designed to be anti-slip at least in one direction. This serves to prevent the inner protective layer from slipping on the prosthesis and thus also stops the sensor layer from slipping out of position in the sensor support.

In particular, at least a section of the inner protective layer is made from an anti-slip material. An anti-slip material prevents the sensor support from slipping out of position while the prosthesis is in use. The anti-slip material interacts frictionally with the material of the prosthesis.

Alternatively or additionally, at least a section of the inner protective layer is made from a slip-favouring material. A slip-favouring material enables the sensor support to be pulled over the prosthesis more easily. In such a structure, the slip-favouring material interacts with the prosthesis in sliding manner at least in one direction.

In particular, the inner protective layer is designed to favour slipping at least in one direction. This makes it easier to pull the sensor support over the prosthesis and prevents the formation of creases in the sensor support. The slip-favouring material may be applied to support material as a coating. In this arrangement, the support material may be dimensionally stable and the coating may be designed to favour slipping. As described previously, for example, a synthetic leather coated with vinyl may be used as the support material.

The inner protective layer preferably has a profile structure for enhancing its anti-slip or slip-favouring property. In this arrangement, the profile structure of the inner protective layer interacts with the prosthesis to promote sliding in one direction and interacts with the prosthesis frictionally in another direction.

Alternatively or additionally, the inner protective layer is designed to inhibit slipping at least in one direction. This prevents the inner protective layer from slipping out of position which in turn stops the sensor layer in the sensor support from slipping out of position.

In particular, as described herein the profile structure is embossed, which has the effect of further amplifying the properties of slip stability in the sensor section without making it more difficult to arrange the sensor support on an item of clothing.

The sensor layer is preferably embedded between the outer protective layer and the inner protective layer, so that any formation of creases in the sensor layer due to mechanical load is prevented.

In particular, the sensor layer is embedded separably between the outer protective layer and the inner protective layer in such manner that is can be replaced. This makes it possible to clean the sensor support without involving the sensors themselves in the cleaning operation.

More preferably, the sensor layer is arranged in the sensor section with material bonding. In this arrangement, a first side of the sensor layer is bonded to the sensor section adhesively, for example, so that potential shearing forces can be neutralised through the outer protective layer or the inner protective layer and are not transferred to the sensor layer.

Preferably, a control apparatus is provided and is connected electrically to the sensors. The control device receives the electrical signals generated by the sensors and is able to process them further. The sensors are connected to the control apparatus via electrical wires. The electrical wires may either be separate wires or they may be electrically conductive threads which have been worked into one of the knitted structures.

More preferably, the control apparatus is connected separably to the sensors of the sensor layer. For this purpose, the control apparatus may include a connection section on which the electrical wires are arranged so as to be detachable or separable. The control apparatus may optionally include various control programs, which may be adapted to reflect the structural arrangement of the sensors in the sensor layer, for example. Additionally, a sensor layer may be operated with different control apparatuses. The use of a sensor layer according to the invention in or on a sensor support according to the invention may be conceivable for various control apparatuses, and accordingly the sensor layer and/or the sensor support can be combined as a module with a range of modular control apparatuses.

The control apparatus may preferably be attachable to the prosthesis part. In this way, the control apparatus may be arranged on the prosthesis part or the prosthesis separately from the sensor support. The prosthesis part is the prosthesis shaft, for example, with which a firm attachment of the control apparatus can be created. This embodiment and those described hereinafter are also inventive and have industrial applicability independently of the other features of the sensor support.

The control apparatus is preferably attachable to the prosthesis part in such manner that it can be separated again. In this way, the control apparatus is held securely and the replacement of the control apparatus is made simpler for the prosthesis user.

The control apparatus is preferably attachable to the prosthesis part by means of a magnetic holder. The separable attachment of the control apparatus is further improved thereby.

Further advantages, features and details of the invention may be discerned from the following description, in which the invention is described with reference to the drawing and exemplary embodiments. Enumerations such as first, second, third or similar serve solely to identify the components.

The list of reference numerals is an integral part of the disclosure, as are the technical content of the claims and figures.

The descriptions of the figures are interrelated and unified. The same reference numerals denote identical components, reference numerals with different indices indicate functionally equivalent or similar components.

Document US 2016/206242 A1 discloses a sensor apparatus in a flexible and stretchable item of clothing, in which the sensors arranged therein capture physiological parameters of the skin or fabric covered by the item of clothing. The sensors arranged in the item of clothing may also capture parameters such as force or pressure exerted on or against a fabric or an area of skin below it. The sensor apparatus is equipped with pressure sensors and wires which are made from conductive fabric material and are worked directly into the item of clothing.

The object of US 2016/206242 A1 may appear to be a comparable sensor support, which is the reason for which the abovementioned document is cited here. However, there is no link between this known document and the statement of the problem, or the solution to the problem, that would motivate the person skilled in the art to make use of the teaching of this document, because the object of said document is entirely unrelated to the field of prosthetics.

### IN THE DRAWING

FIG. 1 is a perspective view of a sensor support according to the invention,

FIG. 2 is a top view of a sensor layer of the sensor support of FIG. 1,

FIG. 3 shows the sensor layer of FIG. 2 in the sensor section of the sensor support of FIG. 1 along cross sectional line III,

FIG. 4 is a top view of an inner protective layer of the sensor section in the sensor support of FIG. 1,

FIG. 5 is a top view of an outer protective layer of the sensor section in the sensor support of FIG. 1,

FIG. 6 is a side view of the sensor support of FIG. 1 with a control apparatus, and

FIG. 7 is a perspective view of the sensor support of FIG. 1 with a control apparatus on a prosthesis.

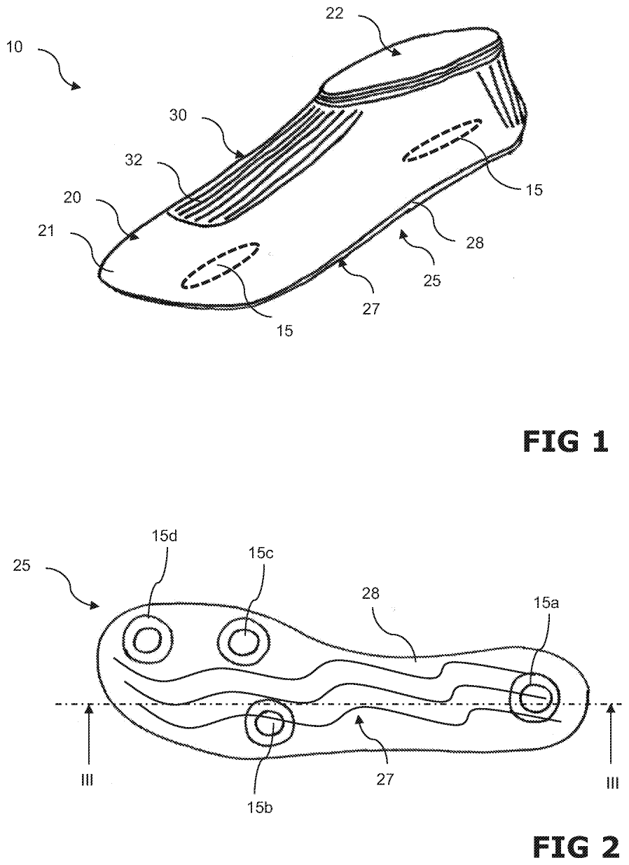

FIG. 1 shows the sensor support 10 for arrangement on a prosthesis or a prosthesis part, wherein the prosthesis is for example an artificial foot. The sensor support 10 comprises a main part 20, consisting of a knitted fabric tube 21 which is closed at one end (the front) of the tube and has a fabric tube opening 22 at an end of the tube opposite the closed end (the rear). The main part 20 is equipped with a sensor section 25 having sensors 15 (only shown symbolically here) and a holding section 30 with a first knitted structure 32, which allows a secure hold of the main part 20 on the prosthesis part. The sensor section 25 has a knitted structure 27 which is different from the first knitted structure 32, and which allows a secure positioning of the sensors 15 relative to the prosthesis part. A sensor layer 28 in which the sensors 15 are disposed in a structured and systematic way is arranged in the sensor section 25. The knitted structure 27 is made from a durable synthetic fibre, whose denier is designed to ensure long-term protection of the sensors 15. The first knitted structure 32 of the holding section has a denier which is different from the denier of the knitted structure 27 of the sensor section.

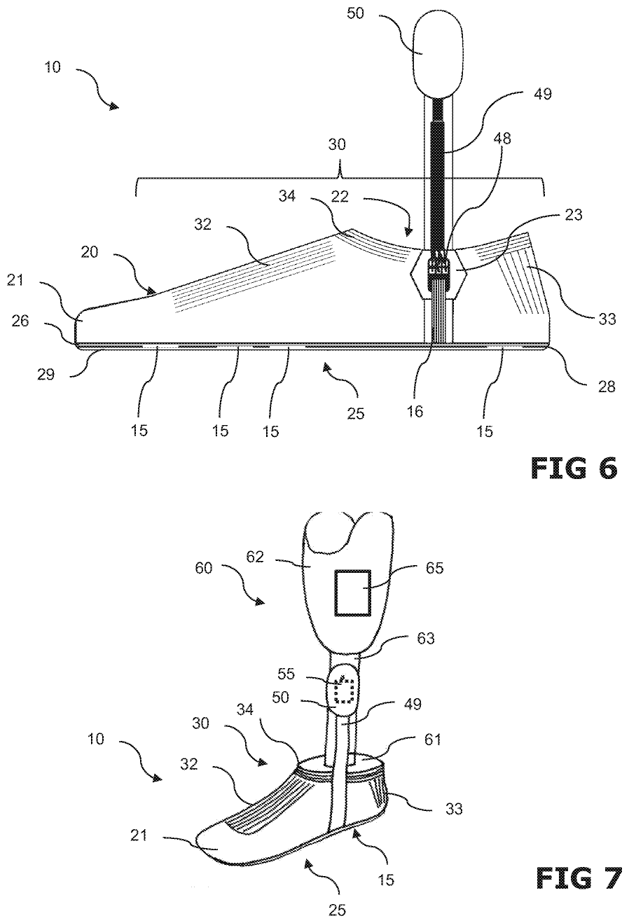

FIG. 2 shows the sensor layer 28 arranged in the sensor section 25 of the sensor support 10. In this exemplary embodiment, the sensor layer 28 has the form of the sole of a foot. In this arrangement, the sensors 15 are disposed in different positions of the sensor layer 28. Four sensors 15 are represented, wherein one sensor 15a is arranged in the heel area, two sensors 15b and 15c are arranged in the area of the ball of the foot, and one sensor 15d is arranged on the foot sole in the area of the toes. The sensors 15 are held in the sensor section with the aid of the knitted structure 27. The denier of the knitted structure 27 has a value of about 0.8 mm. The sensors 15 are pressure sensors and/or force sensors which generate an electrical signal proportional to a pressure force when the pressure force is applied. The sensors 15 are connected to an electrical wire (see FIG. 6) with which the electrical signal can be transported of the sensor layer 28.

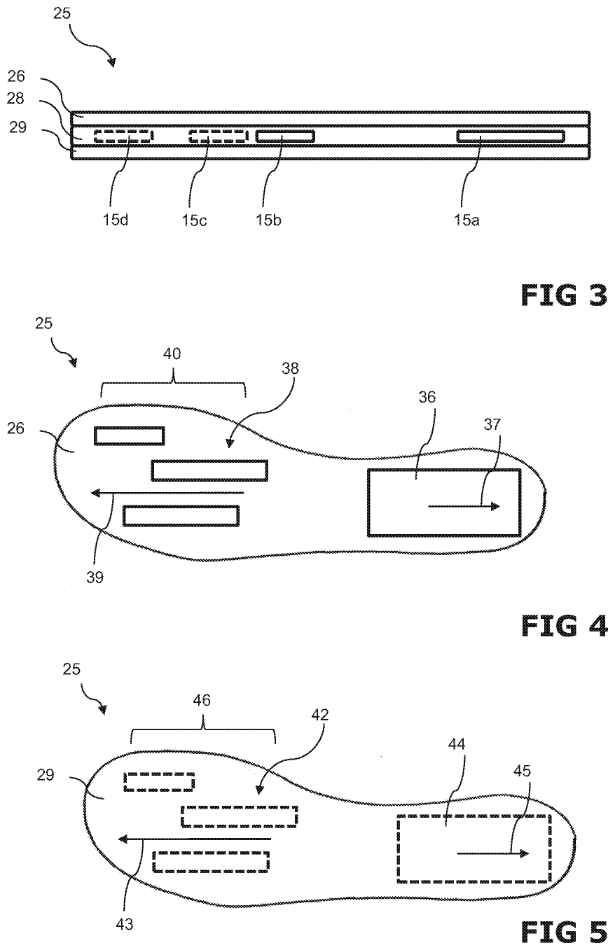

FIG. 3 shows the sensor section 25, which has a multilayer structure. The sensor layer 28 with the sensors 15 embedded therein has an inner protective layer 26 on one side and an outer protective layer 29 on the opposite side. The two protective layers 26 and 29 protect the sensor layer 28 which is positioned between them, so that formation of creases in the sensor layer 28 may be prevented. At least the periphery of the sensor layer 28 is bonded adhesively to the outer protective layer 29.

FIG. 4 shows the inner protective layer 26 of the sensor section 25. In the use case, this inner protective layer 26 faces towards a leg prosthesis and is therefore in contact with the prosthesis part. A portion of the inner protective layer 26 is made from an anti-slip material 36, which prevents the inner layer 26 from slipping out of position in a first direction 37. In the case of a foot sole, this anti-slip Material 36 is arranged in the heel area, for example. A portion of the inner protective layer 26 is made from a slip-favouring material 38, which improves slipping of the inner protective layer 26 in a second direction 39. In such an arrangement, the first direction 37 may ideally extend in the opposite direction to the second direction 39. In the case of a foot sole, this slip-favouring material 38 is arranged in the region of the ball of the foot, for example. The slip-favouring material 38 is constructed as a multipart profile structure 40, wherein the parts thereof are at a structured, functional distance from each other. For example, the anti-slip material and the slip-favouring material are made from the same material which exhibits the same property in different directions.

FIG. 5 shows the outer protective layer 29 of the sensor section 25. In the use case, this outer protective layer 29 faces away from the prosthesis and is therefore in contact with the ground on which the prosthesis is supported in the use case. A portion of the outer protective layer 29 is made from an anti-slip material 42, which prevents the outer layer 29 from slipping on the ground in a first direction 43. In the case of a foot sole, this anti-slip Material 42 is arranged in the region of the ball of the foot, for example. A portion of the outer protective layer 29 is made from a slip-favouring material 44, which improves slipping of the outer protective layer 29 in a second direction 45. In such an arrangement, the first direction 43 may ideally extend in the opposite direction to the second direction 45. In the case of a foot sole, this slip-favouring material 44 is arranged in the heel region, for example. The anti-slip material 42 is constructed as a multipart profile structure 46, wherein the parts thereof are at a structured, functional distance from each other.

For example, the anti-slip material and the slip-favouring material are made from the same material which exhibits the same property in different directions.

FIG. 6 shows the sensor support 10, which in this case is additionally connected electrically to a control apparatus 50. The main part 20 comprises the fabric tube 21 with a fabric tube opening 22. The main part 20 has a holding section 30 which has a second knitted structure 33 besides it first knitted structure 32, wherein the two are constructed differently. The denier of the first knitted structure 32 has a value of about 0.3 mm. The second knitted structure 33 is dimensionally stable and comprises the heel part of an artificial foot arranged in the fabric tube 21 in the use case. The denier of the second knitted structure 33 has a value of about 1 mm. The holding section further has a third knitted structure 34, which is of elastic construction and has denier value about 0.6 mm. The knitted structures 33, 33, 34 are knitted with synthetic fibre strands. The sensor section 25 comprises the sensors 15, which are connected electrically to the control apparatus 50. For this purpose, each sensor 15 is connected to an electrical wire 16, so that each sensor 15 is connected to the control apparatus 50 to enable the exchange of electrical signals. The electrical signals are generated when an external pressure force is detected at the respective sensor 15, and are then forwarded by said sensor 15 to the control apparatus 50, where they are processed further. The electrical wires 16 initially run inside the sensor layer 26, they then exit the sensor layer 26 and run along the fabric tubes 21 towards the fabric tube opening 22. The fabric tube 21 includes a connecting section 23 (interface) in the area of the fabric tube opening 22. An interface 48 is arranged on the connecting section 23, at which interface the electrical wires 16 are connected separably to the control apparatus 50. The control apparatus 50 is arranged on a conductive strip 49 (ribbon conductor) which contains electrical wires. The electrical wires of the conductive strip 49 connect the control apparatus 50 to the interface 48 so that the electrical signals from the sensors 15 can be exchanged with the control apparatus 50. The conductive strip 49 is separable from the interface 48, which means the control apparatus 50 is connected separably to the sensors 15.

FIG. 7 shows the sensor support 10 on an artificial foot 61 of a prosthesis 60. The artificial foot 61 is connected mechanically to a prosthesis shaft—in a manner known per se—by a connecting element 62. When the artificial foot 61 is arranged in the sensor support 10, it is inserted in the fabric tube 21 and held in place in a force-fit by the knitted structures 32, 33, 34 of the holding section 30. The sole of the artificial foot 61 lies flush against the sole section 25 of the sensor support 10. Accordingly, when in use the sole of the artificial foot 61 rests indirectly on the sensors 15 of the sensor support. The control apparatus 50 is attached separably to the connecting element 63 of the prosthesis 60 by means of a magnetic holder 55.

The magnetic holder 55 enables the control apparatus 50 to become detached from the connecting element 63 relatively easily and non-destructively, if a user trips inadvertently somewhere, for example.

The control apparatus 50 is connected electrically to stimulators 65, which are or can be arranged on a prosthesis shaft 62 of the prosthesis 60. In this way, it is guaranteed that the electrical signals from the sensors 15 are processed in the control apparatus 50 and can be used to excited the respective stimulators 65.

### LIST OF REFERENCE NUMERALS s

10 Sensor support
15 Sensors
16 Electrical wires for 15
20 Main part
21 Fabric tube
22 Fabric tube opening
23 Connecting section
25 Sensor section
26 Inner protective layer
27 Knitted structure from 25
28 Sensor layer
29 Outer protective layer
30 Holding section
32 First knitted structure from 30
33 Second knitted structure from 30
34 Third knitted structure from 30
36 Anti-slip Material
37 First direction
38 Slip-favouring material
39 Second direction
40 Profile structure from 26
42 Anti-slip Material
43 First direction
44 Slip-favouring material
45 Second direction
46 Profile structure from 29
48 Interface
49 Conductive strip
50 Control apparatus
55Magnetic holder
60 Prosthesis
61 Artificial foot
62 Prosthesis shaft
63 Connecting element
65 Stimulators

1. A Sensor support (10) for arrangement on a prosthesis, in particular an artificial hand or an artificial foot, comprising a main part (20) for accommodating sensors (15), wherein the main part (20) has a sensor section (25) with sensors (15), and a holding section (30) for holding the main part (20) on a prosthesis part, characterized in that the main part (20) has a knitted fabric tube (21), and wherein the holding section (30) has a first knitted structure (32) which enables securely seated holding of the main part (20) on the prosthesis part, and that the sensor section (25) has knitted structure (27) different therefrom, which enables secure positioning of the sensor (15) relative to the prosthesis part.

2. The sensor support according to claim 1, characterized in that besides its first knitted structure (32) the holding section (30) has a second knitted structure (33), which is designed differently from the first knitted structure (32) and in particular is of dimensionally stable design to enclose a prosthesis part.

3. The sensor support according to claim 2, characterized in that besides its first knitted structure (32) the holding section (30) has a third knitted structure (34), which is preferably designed differently from the first knitted structure (32) and the second knitted structure (33), and in particular is of elastic construction.

4. The sensor support according to claim 2 or 3, characterized in that the first knitted structure (32) of the holding section (30) has a first denier and the second knitted structure (33) of the holding section (30) has a second denier, and the third knitted structure (34) of the holding section (30) preferably has a third denier.

5. The sensor support according to any one of claims 1 to 4, characterized in that the knitted structure (27) of the sensor section (25) has a denier which is different from the first denier of the holding section (30).

6. The sensor support according to any one of claims 1 to 5, characterized in that at least one of the knitted structures (27, 32, 33, 34) is produced from a durable synthetic fibre.

7. The sensor support according to any one of claims 1 to 6, characterized in that the sensor section (25) is of multilayer construction and includes a sensor layer (28) in which the sensors (15) are embedded, wherein the sensors preferably comprise at least one pressure sensor and/or at least one force sensor.

8. The sensor support according to any one of claims 1 to 7, characterized in that an outer protective layer (29) which in the used state faces away from a prosthesis part is provided on the sensor section (25) in addition to the knitted structure (27), and an inner protective layer (26) is preferably provided facing towards the prosthesis part.

9. The sensor support according to claim 8, characterized in that the outer protective layer (29) and/or the inner protective layer (26) is/are designed to inhibit slipping at least in one direction, in particular at least a portion thereof is made from anti-slip material and/or at least a portion thereof is made from material which favours slipping at least in one direction, in particular at least a portion thereof is made from slip-favouring material and preferably has a profile structure for enhancing the anti-slip or slip-favouring property.

10. The sensor support according to either of claim 8 or 9, characterized in that the sensor layer (28) is embedded between the outer protective layer (29) and the inner protective layer (26).

11. The sensor support according to any one of claims 7 to 10, characterized in that the sensor layer (28) is arranged with a material bond in the sensor section (25).

12. The sensor support according to any one of claims 1 to 11, characterized in that a control apparatus (50) is provided which is electrically connected to the sensors (15).

13. The sensor support according to claim 12, characterized in that the control apparatus (50) is connected separably to the sensors (15) of the sensor layer (28).

14. The sensor support according to either of claim 12 or 13, characterized in that the control apparatus (50) can be attached preferably separably to the prosthesis part.

15. The sensor support according to claim 14, characterized in that the control apparatus (50) can be attached to the prosthesis part by means of a magnetic holder (55).
